(19) United States
(12) Patent Application Publication
(10) Pub. No.: US 2010/0222630 A1
(43) Pub. Date: Sep. 2, 2010

(54) METHOD AND APPARATUS FOR LOW FREQUENCY INDUCTION THERAPY FOR THE TREATMENT OF URINARY INCONTINENCE AND OVERACTIVE BLADDER

(75) Inventors: Shane MANGRUM, Ammon, ID (US); Daniel BURNETT, San Francisco, CA (US)

Correspondence Address: LEVINE BAGADE HAN LLP, 2400 GENG ROAD, SUITE 120, PALO ALTO, CA, 94303, US

(73) Assignee: EMKINETICS, INC., Santa Cruz, CA (US)

(21) Appl. No.: 12/695,087

(22) Filed: Jan. 27, 2010

Related U.S. Application Data
(62) Division of application No. 11/332,797, filed on Jan. 17, 2006, now abandoned.

(51) Int. Cl. A61N 2/04; A61N 2/02

(52) U.S. Cl. 600/14; 600/13

(57) ABSTRACT

Current treatment options for Overactive Bladder and Urinary Incontinence include exercise and behavioral modifications, pharmacological therapies, surgical intervention, and neuromodulation. Although each of these treatments is used in the treatment of individuals with these conditions, each has severe limitations. Building on the limitations of existing therapies, and with the distillation of lessons learned from the field of pulsed electric stimulation, the present invention employs Low Frequency Induction Therapy for the delivery of an effective, cost efficient, noninvasive alternative to available treatment options. The device of the present application allows for consistent, user-friendly modulation of the pudendal nerve and the sacral plexus, via pulsed electromagnetic stimulation of the posterior tibial nerve, on an outpatient basis. The device has two primary components: a programmable Logic Controller (LC), which generates the required current, and a Conductive Wrap (CW), through which the current is channeled in generating the pulsed electromagnetic fields.

### CROSS-REFERENCE TO RELATED APPLICATIONS

This application is a divisional of U.S. patent application Ser. No. 11/332,797, filed Jan. 17, 2006, the content of which is incorporated herein by reference in its entirety.

### BACKGROUND OF THE INVENTION

The present invention relates to the field of medical devices, in particular therapeutic intervention devices for treatment and prevention of urinary incontinence (UI) and overactive bladder (OAB) with the use of Low Frequency Induction Therapy (LoFIT).

The OAB and UI market in the United States is well over a $12 billion a year industry. It affects over 16% of all Americans, for a total U.S. market of approximately 34 million men and women each year. Due to social stigmas attached to OAB and UI, as well as misunderstanding of the signs and symptoms associated with OAB and UI, only 40% of those affected (13.6M) seek treatment. Of those 13.6 million individuals, nearly 30% are unsatisfied with their current therapy.

The use of pulsed electromagnetic stimulation (PES) has been well established as a beneficial therapy in a variety of medical applications. The scientific principle behind this technology is that an electric current passed through a coil will generate an electromagnetic field. These fields, in turn, have been shown to induce current within conductive materials placed within the field. When applied to the human body, pulsed electromagnetic stimulation has been found to be an effective method of stimulating nerves resting within the electromagnetic field. Building on recent data, which highlights the beneficial effects of invasive, needle-based electrostimulation (ES) of the posterior tibial nerve in individuals with OAB and UI, there is strong evidence for the treatment of these ailments with the use of LoFIT. In particular, ES has been found to modulate bladder dysfunction through its action on the pudendal nerve and the sacral plexus which provides the major excitatory input to the bladder.

Current treatment options for OAB and UI are exercise and behavioral modifications, pharmacological therapies, surgical intervention, and neuromodulation. Although each of these treatment options targets the UI and OAB populations, each has severe limitations.

Exercise and behavioral modifications often require patients to adhere to stringent routines, including scheduled voiding, maintenance of a bladder diary, and intense exercise regiments. While this may be a viable option for a small group of highly dedicated individuals, its daily impact on one's life makes it an unattractive option for most individuals.

Pharmacological intervention is the most widely prescribed therapy for OAB and UI. Unfortunately, as with the ingestion of any chemical, patients are often subject to side effects from their drug therapy. This is especially detrimental in older and elderly patient populations where interaction with other prescribed medications can have adverse effects. Further, there is a high rate of dissatisfaction, approximately 30%, amongst individuals using pharmacological treatment.

Surgical intervention is an extremely invasive treatment and often results in the long-term, and in some cases permanent, requirement for catheterization. The high expense of these procedures, coupled with the negative impact the procedures have on the patients quality of life, make this an option only when all other treatment options have been exhausted.

Neuromodulation is another treatment alternative for OAB and UI patients. Sacral nerve stimulation (SNS) has shown itself to be an effective treatment option for those with OAB or UI. However, the procedure requires the permanent implantation of an electrical stimulation device in the patient. One estimate puts the cost at nearly $14,000 with additional routine care costs of $593 per patient per year. Additionally, SNS's risk of battery failure, implant infection, and electrode migration, lead to a high reoperation rate and make this procedure unattractive.

More recently, the introduction of a posterior tibial nerve stimulator, often referred to as SANS, has shown itself to be another neuromodulation alternative. Yet as is the case with other forms of neuromodulation, this system is invasive in its nature. It requires the insertion of a needle two inches into the patient's ankle region in order to stimulate the posterior tibial nerve. As well, it requires a minimum of 12 sessions for initial treatment, with the possibility of additional sessions needed for maintenance. Despite its high cost and invasive nature, though, an abundance of published peer-reviewed clinical trials demonstrate the safety and efficacy of the SANS therapy.

### REVIEW OF THE PRIOR ART

U.S. Pat. No. 6,941,171 describes a method and system for treatment of incontinence, urgency, frequency, and/or pelvic pain includes implantation of electrodes on a lead or the discharge portion of a catheter adjacent the perineal nerve(s) or tissue(s) to be stimulated. Stimulation pulses, either electrical or drug infusion pulses, are supplied by a stimulator implanted remotely, and through the lead or catheter, which is tunneled subcutaneously between the stimulator and stimulation site. This device, while holding some therapeutic potential, is invasive in its delivery and requirement for implantation of device components.

U.S. Pat. No. 5,984,854 describes a method for treating urinary incontinence which consists of delivering a train of current pulses through one or more magnetic stimulation coils to induce a train of magnetic flux pulses, which then induce an eddy current within the body, thereby to stimulate a group of pelvic floor muscles, the pudendal nerve, the external urethral sphincter, or the tibial nerve. While this device describes the employment of pulsed electromagnetic fields in the treatment of urinary incontinence, the application does not contemplate the use of any specific component to facilitate the placement of the magnetic coils over a targeted region of the body. That is, the application describes holding a coil over an intended region of the body, but does not contemplate the use of ergonomic wraps or other means for allowing an untrained user to apply the intended treatments.

U.S. Pat. No. 6,735,474 describes a method and system for treatment of incontinence and/or pelvic pain includes the injection or laparoscopic implantation of one or more battery- or radio frequency-powered microstimulators beneath the skin of the perineum and/or adjacent the tibial nerve. The devices are programmed using radio-frequency control via an external controller that can be used by a physician to produce patterns of output stimulation pulses judged to be efficacious by appropriate clinical testing to diminish symptoms. The stimulation program is retained in the microstimulator device or external controller and is transmitted when commanded to start and stop by a signal from the patient or caregiver. Again, this application involves the implantation of device components and is thus relatively invasive in nature.

U.S. patent application 20050171576 describes an electro-nerve stimulation apparatus includes a pulse generator, a first electrically conductive, insulated lead wire, a second electrically conductive, insulated lead wire, an electrically conductive transcutaneous electrode and an electrically conductive percutaneous needle electrode. Connected to one end of the first and second lead wires is a connector for electrically coupling with the pulse generator. The transcutaneous electrode is operably connected to the other end of the first lead wire. An electrically conductive adaptor is secured to the other end of the second lead wire for electrically coupling to the terminal end of the percutaneous needle electrode. The lead wire set includes a single-use mechanism adapted to effectively discourage reuse of the electrodes. In use, the transcutaneous electrode is adhered to the patient's skin distal from the desired internal stimulation site. The percutaneous needle electrode is inserted through the skin in proximity to the desired internal stimulation site. The pulse generator is activated to pass current pulses between the transcutaneous electrode and the percutaneous needle electrode through the internal stimulation site. This device employs pulsed electric stimulation, and does not provide for the delivery of pulsed electromagnetic stimulation. Moreover, the device does not contemplate mechanism for facilitating use of the device by an untrained user (such as a wrap with markings to facilitate correct placement of the appliance).

### SUMMARY OF THE INVENTION

The use of pulsed electromagnetic stimulation (PES) has been well established as a beneficial therapy in a variety of medical applications. The scientific principle behind this technology is that an electric current passed through a coil will generate an electromagnetic field. These fields, in turn, have been shown to induce current within conductive materials placed within the field. When applied to the human body, pulsed electromagnetic stimulation has been found to be an effective method of stimulating nerves resting within the electromagnetic field

Building on recent data, which highlights the beneficial effects of invasive, needle-based electrostimulation (ES) of the posterior tibial nerve in individuals with OAB and UI, there is strong evidence for the treatment of these ailments with the use of the proposed Low Frequency Induction Therapy System (LoFIT ™ System). In particular, ES has been found to modulate bladder dysfunction through its action on the pudendal nerve and the sacral plexus which provides the major excitatory input to the bladder. ES, and now the LoFIT System, have been built upon animal and human studies demonstrating how electrical stimulation of the pelvic and sacral nerves influence bladder emptying and provide neuromodulation therapies for OAB and UI. While the LoFIT ™ System is capable of generating an electrical stimulus identical to that found in ES, it is completely non-invasive and can be applied by an untrained user.

The LOFIT ™ System, consists of two components: a programmable Logic Controller (LC), which generates the required current, and a Conductive Wrap (CW), through which the current is channeled in generating the pulsed electromagnetic fields. This novel technology is embodied in its CW which allows for consistent, user-friendly targeting and modulation of the pudendal nerve and the sacral plexus, via the posterior tibial nerve, on an outpatient basis.

The LOFIT ™ System is currently protected by 3 patents pending and one issued patent: U.S. Pat. No. 6,701,185 entitled “Method and apparatus for electromagnetic stimulation of nerve, muscle, and body tissues”. Through benchtop testing, it has demonstrated that the LOFIT ™ System is capable of non-invasively generating electrical currents similar to those found in therapeutic ES.

In its preferred embodiment, the CW is disposed in a flexible material. Said flexible material will be easily placed onto the body of the user for application of the LoFIT. Markings on this component may indicate how the wrap should be positioned on the body, making the device easy to use and user-friendly. The device may be used while ambulatory or in a seated position.

In one embodiment of the device, the CW will contain an array of overlapping coils disposed in the flexible material. The overlapping coils will help to assure accurate targeting of the Tibial Nerve with the therapy in a home healthcare setting. In an alternative embodiment of the device, the CW will contain a Helmholtz coil arrangement disposed in the flexible material for the delivery of LoFIT. The coil arrangement may take any shape, though, so long as the stimulation of the nerve is achieved.

In one embodiment of the device, said CW will be disposed in a material that is intended to be positioned over the ankle. This design will help to stimulate the Tibial Nerve in the region of the ankle in a location that is easily targeted for therapeutic intervention.

In an alternative embodiment of the device, said CW will be disposed in a material that is intended to fit over the knee (or elsewhere along the course of the Sciatic and Tibial Nerve) for the delivery of therapeutic stimulation.

In one embodiment of the device, the LC component is powered by a portable power source (e.g. rechargeable battery) to facilitate mobility with delivery of the intended treatment. In an alternative embodiment of the device, the LC can be powered by a fixed power source (e.g. plugging the device into a traditional outlet in the wall).

The competitive advantages of the LoFIT ™ System include: (1) non-invasive nature of the delivery system, (2) the system does not require the ingestion of potentially harmful chemical compounds, (3) the system is user friendly and capable of being used by an unskilled patient in home healthcare settings, and (4) the system will provide superior therapy while reducing current costs associated with treatment and care of OAB and UI patients.

Despite the name LOFIT, it is important to note that any frequency of stimulation may be delivered by the present invention including, but not limited to, low-frequency, high-frequency, mid-frequency, ultrahigh frequency, etc. Furthermore, non-overlapping coils may also be used to generate the desired field, although overlapping or Helmholtz coils are ideal in their ability to target a broader region and achieve more thorough stimulation.

### BRIEF DESCRIPTION OF THE DRAWINGS

FIG. 1 is a perspective view of the LoFIT System illustrating the components for delivery of Low Frequency Induction Therapy in one embodiment of the present invention with the CW incorporating an array of overlapping coils.

FIG. 2 is a perspective view of the LoFIT System illustrating the components for delivery of Low Frequency Induction Therapy in one embodiment of the present invention with the CW incorporating a Helmholtz coil arrangement.

FIG. 3 is a perspective view of the LoFIT System illustrating the components for delivery of Low Frequency Induction Therapy in one embodiment of the present invention with the CW disposed over the knee for delivery of the intended therapy. In this and other embodiments the overlapping or Helmholtz coils are desirable in order to allow application by an untrained user.

### DETAILED DESCRIPTION OF THE DRAWINGS

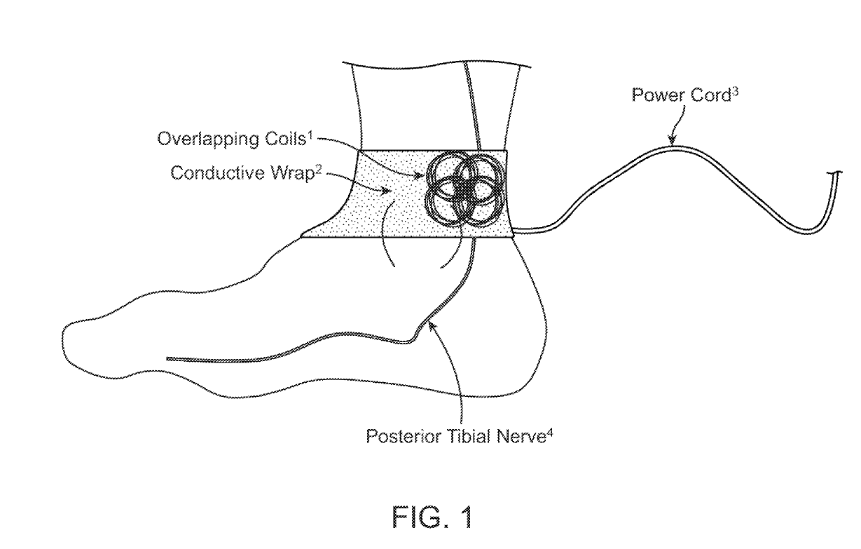

The invention is susceptible to many and various embodiments; those embodiments described below should not be interpreted as restrictive, but rather as merely illustrative of the invention. As illustrated in FIG. 1, one embodiment of the present invention provides a LoFIT System with a CW 2 disposed over the Tibial Nerve 4 in the region of the ankle. The CW 2 is shown incorporating an array of overlapping coils 1.

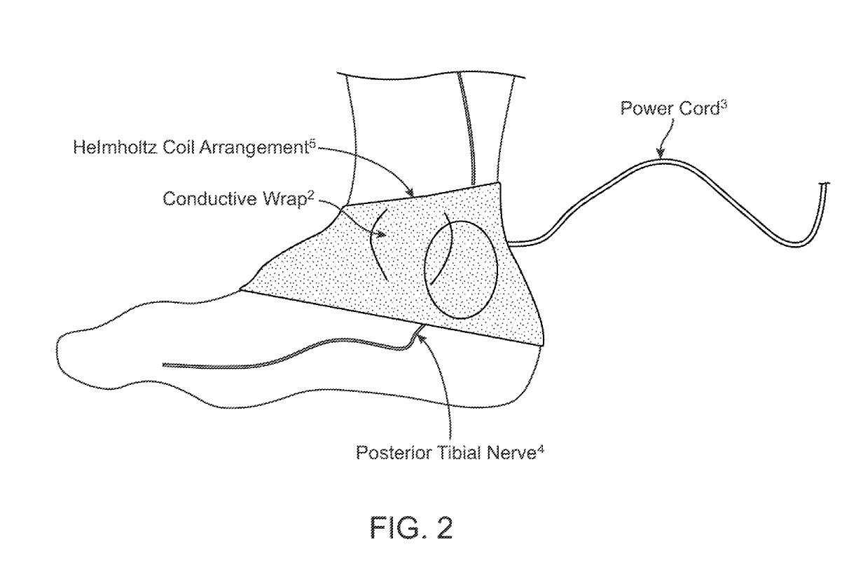

FIG. 2 illustrates an alternative embodiment of the present invention, with the CW 2 incorporating a Helmholtz coil arrangement 5 positioned over the Posterior Tibial Nerve 4 for therapeutic stimulation.

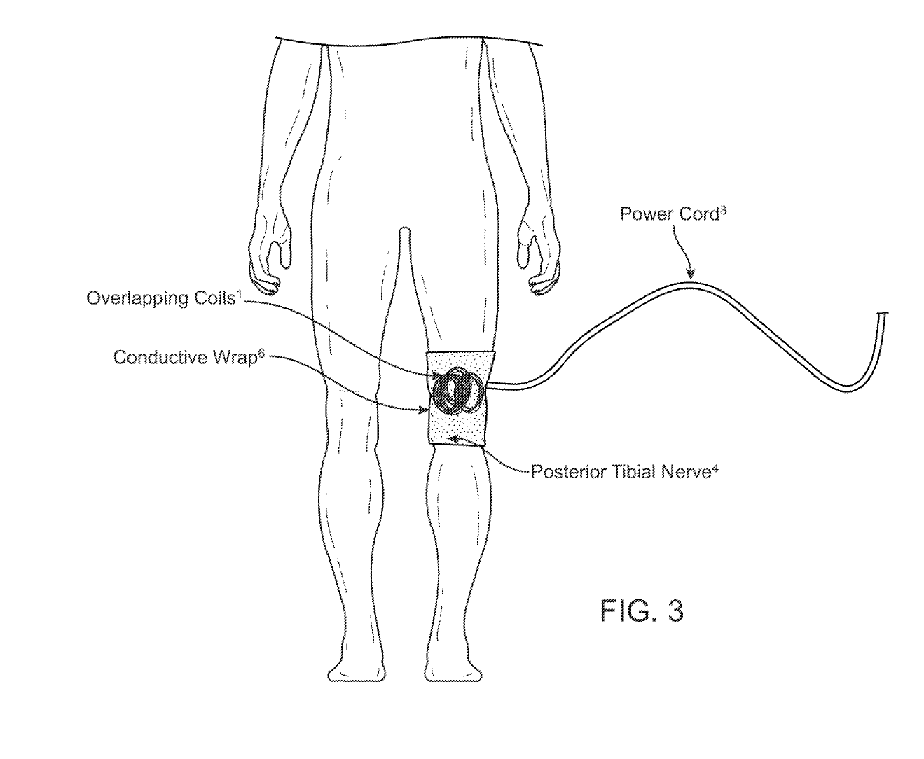

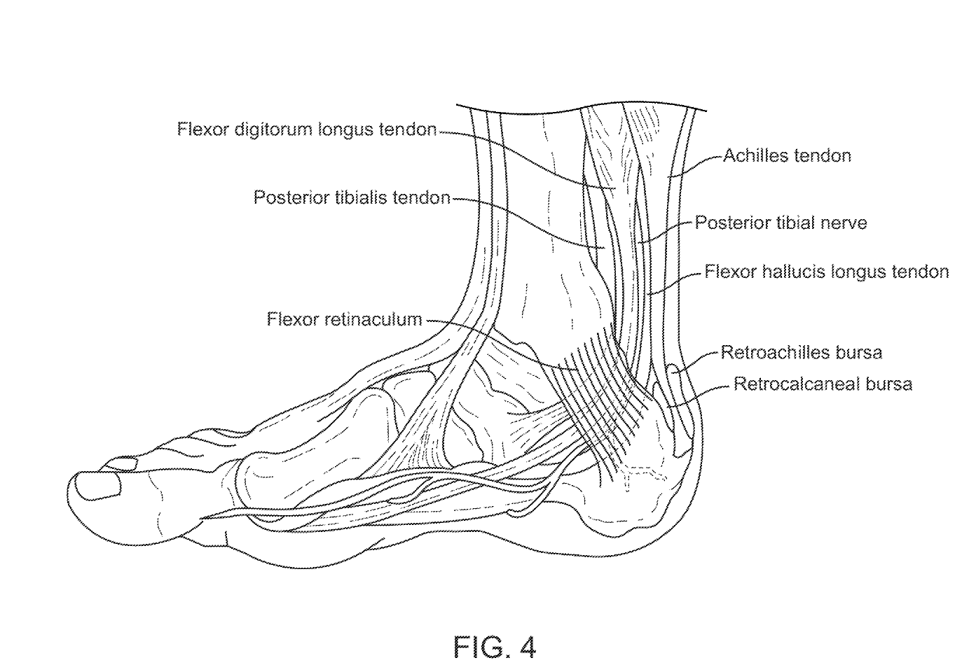

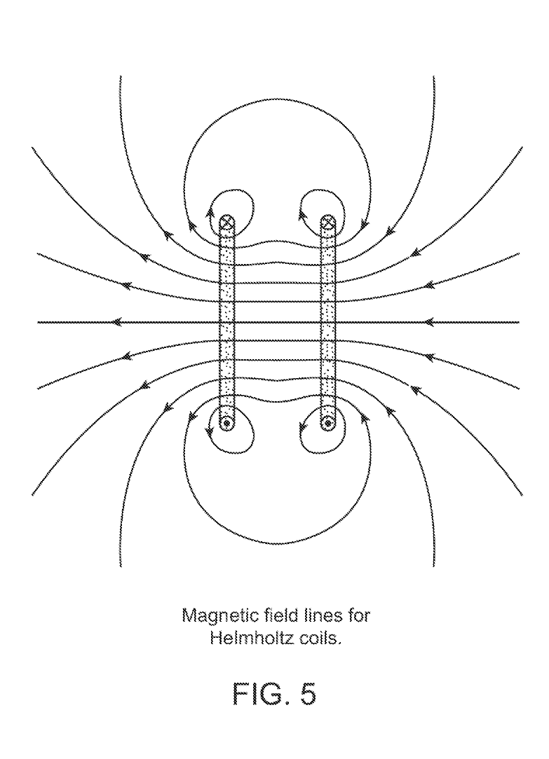

FIG. 3 illustrates an alternative embodiment of the present invention, with the CW 6 positioned now over the popliteal fossa in the region of the knee for stimulation of the Posterior Tibial Nerve 4.

The device of the present invention is unique in that it allows for the stimulation of the tibial nerve in a non-invasive manner, using an ergonomically designed wrap to specifically target the tibial nerve in patients with overactive bladder or incontinence problems.

### DETAILED DESCRIPTION OF THE PREFERRED EMBODIMENTS

Device: Low Frequency Induction Therapy

Indications for Use:

1) Prevention of urinary incontinence (UI)

2) Prevention of overactive bladder (OAB) Methods for Use:

1) A patient with a condition of UI of OAB, for example, will place a conductive wrap disposed in a flexible material over the region of the ankle (or alternatively the knee).

2) The logic controller component of the device will be activated for the directed duration of use, 15-30 minutes in the ideal embodiment. The logic controller may be powered either by a portable power source (e.g. battery) or a fixed power source (e.g. traditional wall outlet).

3) The conductive wrap will be removed from the body when therapeutic stimulation is not being delivered and reapplied as indicated, on a daily basis in the ideal embodiment.

We claim:

1. A method of treating a patient with urinary incontinence or an over-active bladder, comprising:
positioning a conductive coil adjacent to a skin surface of an ankle of the patient such that the conductive coil is in proximity to a tibial nerve positioned through the ankle;
passing a current through the conductive coil such that the tibial nerve is electromagnetically stimulated; and
stimulating a pudendal nerve and a sacral plexus of the patient by electromagnetically stimulating the tibial nerve.

2. The method of claim 1, wherein stimulating comprises stimulating a pudendal nerve via a sacral plexus of the patient by electromagnetically stimulating a posterior tibial nerve.

3. The method of claim 1, wherein the conductive coil is disposed within a flexible conductive wrap.

4. The method of claim 3 wherein positioning comprises placing an array of overlapping coils disposed within the wrap upon the skin surface.

5. The method of claim 3 wherein positioning comprises placing an array of non-overlapping coils disposed within the wrap upon the skin surface.

6. The method of claim 1 wherein passing a current comprises utilizing a programmable logic controller to generate the current.

7. The method of claim 1 wherein passing a current comprises coupling the coil to a portable power source.

8. The method of claim 1 wherein passing a current comprises pulsing the current through the coil to generate a pulsed electromagnetic field for stimulating the tibial nerve.

9. The method of claim 1 wherein stimulating comprises stimulating the posterior tibial nerve between 15 to 30 minutes.

10. The method of claim 1 wherein stimulating comprises stimulating the posterior tibial nerve between 15 to 30 minutes daily.

11. A method of treating a patient with urinary incontinence or an over-active bladder, comprising:
positioning a conductive wrap having at least one conductive coil disposed therewithin adjacent to a skin surface about an ankle of the patient such that the conductive coil is in proximity to a tibial nerve positioned through the ankle;
passing a current through the conductive coil such that the tibial nerve is electromagnetically stimulated; and
stimulating a pudendal nerve or a sacral plexus of the patient by electromagnetically stimulating the tibial nerve.

12. The method of claim 11, wherein stimulating comprises stimulating a pudendal nerve or a sacral plexus of the patient by electromagnetically stimulating a posterior tibial nerve.

13. The method of claim 11, wherein the conductive coil is disposed within a flexible conductive wrap.

14. The method of claim 13 wherein positioning comprises placing an array of overlapping coils disposed within the wrap upon the skin surface.

15. The method of claim 13 wherein positioning comprises placing an array of non-overlapping coils disposed within the wrap upon the skin surface.

16. The method of claim 11 wherein passing a current comprises utilizing a programmable logic controller to generate the current.

17. The method of claim 11 wherein passing a current comprises coupling the coil to a portable power source.

18. The method of claim 11 wherein passing a current comprises pulsing the current through the coil to generate a pulsed electromagnetic field for stimulating the tibial nerve.

19. The method of claim 11 wherein stimulating comprises stimulating the tibial or sciatic nerve between 15 to 30 minutes.

20. The method of claim 11 wherein stimulating comprises stimulating the tibial or sciatic nerve between 15 to 30 minutes daily.

21. The method of claim 11 wherein positioning comprises placement of the conductive wrap about an ankle of a patient according to markings on the wrap which provide user friendly guidance for placement.

22-30. (canceled)
